(19) United States
(12) Patent Application Publication
(10) Pub. No.: US 2005/0215864 A1
(43) Pub. Date: Sep. 29, 2005

(54) Retracting and dissecting apparatus for surgical operation, and minimal incision penile augmentation using the same

(76) Inventor: Su-Yeon Jang, Seoul (KR)

Correspondence Address: BLACKWELL SANDERS PEPER MARTIN LLP, 720 OLIVE STREET, SUITE 2400, ST. LOUIS, MO, 63101, US

(21) Appl. No.: 10/966,573

(22) Filed: Oct. 15, 2004

(30) Foreign Application Priority Data
Mar. 24, 2004 (KR) 10-2004-0020010

(51) Int. Cl. A61B 17/32

(52) U.S. Cl. 600/217

(57) ABSTRACT

A retracting and dissecting apparatus designed to allow easy and safe retraction during a surgical operation. The apparatus comprises first and second dissecting members provided at one end thereof with grips, at the other end thereof with first and second plate-shaped dissecting portions, and at the center thereof with fastening holes, respectively, a fastening member to join the fastening holes of the first and second dissecting members to each other such that the first and second dissecting members can rotate in a predetermined range, and a position holding member between the center of the first and second dissecting members and the grips of the first and second dissecting members to hold the first and second dissecting members. The dissecting portions have plate shapes, preventing the dissected portion of the patient from being damaged, and ensuring a more safe dissection. Minimal incision penile augmentation using the retracting and dissecting apparatus utilizes a small incision thereby reducing suturing time, operation time, and side effects.

### CROSS-REFERENCE TO RELATED APPLICATIONS

This application claims the benefit of Korean Patent Application No. 10-2004-0020010 filed Mar. 24, 2004.

### BACKGROUND OF THE INVENTION

1. Field of the Invention

The present invention relates to a retracting and dissecting apparatus for a surgical operation, and minimal incision penile augmentation using the same and, more particularly to a retracting and dissecting apparatus for a surgical operation designed to allow easy and safe retraction without damaging a dissected portion of a human body during the surgical operation after dissection, and minimal incision penile augmentation using the same.

2. Description of the Related Art

Generally, in some cases of a surgical operation in a hospital, a prosthetic implant is inserted through a dissected portion after dissecting around an incised portion, incision is carried out through the dissected portion, or dissection is performed for the purpose of medical treatment.

As examples of such a surgical operation, there are breast augmentation or penile augmentation.

Penile augmentation is a surgical operation in which some portion of a suspensory ligament connecting a pubic bone and a penis concealed in a human body is incised, thereby increasing the length of the penis exposed outside of the body.

Although the length of the penis concealed within the body differs from person to person, the portion of the penis concealed within the body is generally equal to the length of the penis exposed to the outside of the body.

As one of the surgical operations to increase the length of the penis, a suspensory ligament incision method may be used in which some portion of the suspensory ligament connecting the lower portion of the pubic bone and some portion of the penis concealed in the body is incised to allow the penis to be exposed outside of the body, thereby increasing the apparent length of the penis.

The suspensory ligament acts to maintain an erect penis upwardly, and to hold erectile tissue in the body.

Although incision lengths differ from person to person, it is generally appropriate to incise the suspensory ligament by a length of 2˜5 cm. At this time, when the suspensory ligament is excessively incised, side effects causing the erect penis to be abnormally low may result.

The suspensory ligament incision method uses a direct incision method in which after incising some portion of the lower abdomen in order to expose the suspensory ligament, an exposed portion of the suspensory ligament is incised, or a dissection method in which, after incising dermal tissue covering an upper portion of the penis at the rear end of the glans penis, and dissecting the dermal tissue and spongy tissue of the penis using a dissecting apparatus, some portion of the suspensory ligament is incised by a scalpel inserted through the dissected portion.

Although the dissection method has merits in that only slight scarring occurs, as compared with the direct incision method, it requires high attention to detail since the dissected portion can be impaired.

Particularly, a conventional dissecting apparatus for the dissection method has a structure which easily damages the dissected portion. The conventional dissecting apparatus will hereinafter be described in detail with reference to the accompanying drawings.

FIG. 1 is a diagram illustrating a conventional dissecting apparatus for a surgical operation. Referring to FIG. 1, the conventional dissecting apparatus comprises first and second dissecting members 10 and 20 provided at one end thereof with grips 11 and 21, at the other end thereof with blade-shaped dissecting portions 12 and 22, and at the center thereof with fastening holes, and a fastening member 30 inserted through the fastening holes positioned at the center of the first and second dissecting members 10 and 20 to hold the first and second dissecting members 10 and 20 such that the first and second dissecting members 10 and 20 can rotate in a predetermined range about the center thereof.

The first and second dissecting members 10 and 20 do not cross each other, and thus, as a distance between the grips 11 and 21 is increased, a distance between the blade-shaped dissecting portions 12 and 22 is decreased.

That is, as the distance between the grips 11 and 21 is decreased, the distance between the blade-shaped dissecting portions 12 and 22 is increased.

Operation and problems of the conventional dissecting apparatus constructed as described above will now be described as follows with reference to an example of penile augmentation through the incision of the suspensory ligament.

FIG. 2 shows the portion of the human body where penile augmentation is performed.

First, the surgeon incises some portion between dermal tissue 100 and spongy tissue 70 at the rear end of the glans penis, and then, inserts the retracting and dissecting apparatus into the incised portion, with the distance between the blade-shaped dissecting portions 12 and 22 narrowed.

Then, by widening the distance between the blade-shaped dissecting portions 12 and 22, the surgeon gradually dissects the portion between the dermal tissue 100 and the spongy tissue 70 until a suspensory ligament is exposed.

At this time, due to the blade shape of the dissecting portions 12 and 22, the dermal tissue 100 or the spongy tissue 70 can be damaged.

Additionally, since the portion dissected after incision is a portion in which nerves and blood vessels are concentrated, it is necessary to pay close attention during the surgical operation.

When the suspensory ligament 90 is exposed while dissecting the portion between the dermal tissue 100 and the spongy tissue 70, the surgeon incises some portion of the suspensory ligament 90.

Incision of the suspensory ligament 90 is performed with the scalpel inserted through the dissected portion.

At this time, in order to securely insert the scalpel through the dissected portion to the suspensory ligament 90, the distance between the blade-shaped dissecting portions 12 and 22 of the dissecting apparatus is maintained in a widened state. In this state, the surgeon inserts the scalpel into a space, maintained by the blade-shaped dissecting portions 12 and 22, and incises some portion of the suspensory ligament 90 exposed between the blade-shaped dissecting portions 12 and 22.

However, the conventional dissection apparatus has disadvantages in that the distance between the blade-shaped dissecting portions 12 and 22 must be maintained in the widened state, and in that an additional retracting apparatus is required to secure visibility of the suspensory ligament 90 in sight.

For instance, in the operation described above, an assistant is present, and helps the surgeon to secure a suitable view in such a manner that the assistant pulls the penis towards the legs with one hand while pulling the retracting apparatus in the opposite direction as the penis, thereby holding the retracting apparatus.

As such, with the surgeon's view secured, the surgeon incises the suspensory ligament 90, and cauterizes.

As described above, although the bladed-shape of the dissecting portion of the conventional dissecting apparatus has advantages in dissection, the dissected portion can be damaged thereby.

Furthermore, with the conventional dissecting apparatus, the distance between the dissecting portions of the retracting and dissecting apparatus must be maintained in the widened state in order to incise the portion of the body exposed by the dissection using an incising device inserted into the dissected portion. However, since there is no means to maintain the widened state of the dissecting apparatus, there are problems in that the dissected portion can be damaged during the surgical operation, and that the assistant is needed when using other retracting apparatuses.

### SUMMARY OF THE INVENTION

The present invention has been made to solve the above problems, and it is an object of the present invention to provide a retracting and dissecting apparatus for a surgical operation designed to prevent a dissected portion of a human body from being damaged during dissection, and minimal incision penile augmentation using the same.

It is another object of the present invention to provide a retracting and dissecting apparatus for a surgical operation designed to prevent the dissected portion from being damaged, and to allow easy operation without an additional retracting apparatus and an assistant.

In accordance with an aspect of the present invention, the above and other objects can be accomplished by the provision of a retracting and dissecting apparatus comprising a first dissecting member provided at one end thereof with a grip, at the other end thereof with a first plate-shaped dissecting portion, and at the center thereof with a fastening hole; a second dissecting member provided at one end thereof with another grip, at the other end thereof with a second plate-shaped dissecting portion, and at the center thereof with another fastening hole; a fastening member to join the fastening holes of the first and second dissecting members to each other, such that the first and second dissecting members can rotate in a predetermined range about the center thereof, and a position holding member positioned between the center of the first and second dissecting members and the grips of the first and second dissecting members to hold the first and second dissecting members such that the location of the first and second dissecting members does not change unless a user so desires.

In accordance with another aspect of the present invention, the above and other objects can be accomplished by the provision of a retracting and dissecting apparatus comprising a first dissecting member having a grip associated with one end portion thereof and a first plate-shaped dissecting portion associated with an opposite end portion thereof; a second dissecting member having a grip associated with one end portion thereof and a second plate-shaped dissecting portion associated with an opposite end portion thereof; means for pivotally attaching the first and second dissecting members, the first and second dissecting members being rotatable in a predetermined range about the pivot means; and a position holding member for holding the first and second dissecting members at a particular location relative to each other.

### BRIEF DESCRIPTION OF THE DRAWINGS

The foregoing and other objects and features of the present invention will be more clearly understood from the following detailed description taken in conjunction with the accompanying drawings, in which:

FIG. 1 is a diagram illustrating a conventional dissecting apparatus for a surgical operation;

FIG. 2 is a sectional view illustrating the portion of a human body where penile augmentation is performed; and

FIG. 3 is a diagram illustrating a retracting and dissecting apparatus for a surgical operation according to the present invention;

FIG. 4 is a diagram illustrating an example of performing dissection using the retracting and dissecting apparatus according to the present invention; and

FIG. 5 is a detailed view illustrating a holding member, a latching member, and an elastic member of the retracting and dissecting apparatus for the surgical operation according to the present invention.

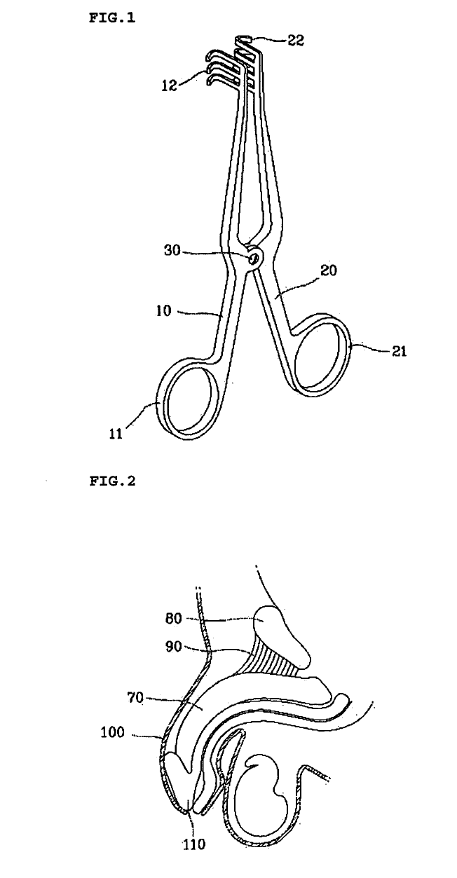

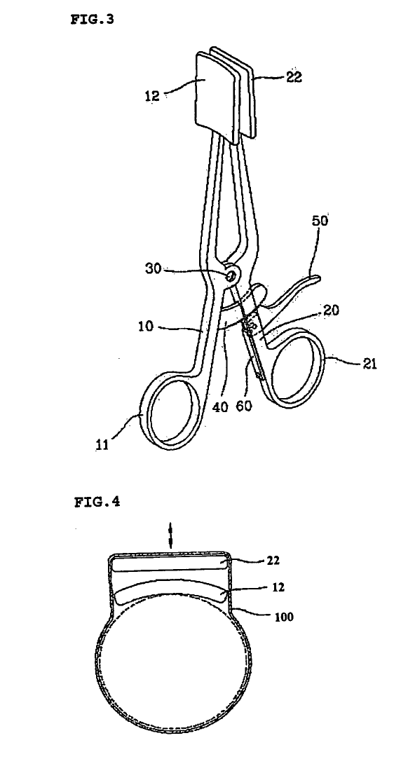

### DESCRIPTION OF THE PREFERRED EMBODIMENTS

Preferred embodiments will now be described in detail with reference to the accompanying drawings wherein like reference numerals refer to like components of a retracting and dissecting throughout the drawings.

Referring to FIG. 3, a retracting and dissecting apparatus according to the present invention comprises a first dissecting member 10 provided at one end thereof with a grip 11, at the other end thereof with a plate-shaped dissecting portion 12, and at the center thereof with a fastening hole; a second dissecting member 20 provided at one end thereof with another grip 21, at the other end thereof with another plate-shaped dissecting portion 22, and at the center thereof with another fastening hole; a fastening member 30 to join the fastening holes of the first and second dissecting members 10 and 20 to each other such that the first and second dissecting members 10 and 20 can rotate in a predetermined range about the center thereof; an arc-shaped holding member 40, extended from an inner surface between the grip 11 and the center of the first dissecting member 10 while protruding through a hole or opening provided between the grip 21 and the center of the second dissecting member 20, and having a plurality of protrusions on an outer surface of the arc-shaped holding member 40; and a latching member 50 rotatably inserted through the hole or opening in the second dissecting members 10 between the center and the grip 21 of the second dissecting member 20 to hold the first and second dissecting members 10 and 20 by engaging with the protrusions of the holding member 40; and an elastic member 60 coupled to an inner surface of the second dissecting member 20 to exert an elastic force to cause the latching member 50 to closely contact the holding member 40. Any suitable fastening member 30 may be used in the practice of the present invention and it is recognized and anticipated that any means for pivotably or rotatably attaching the first and second dissection members 10 and 20 at a predetermined location along their length may likewise be utilized instead of the respective fastening holes and fastening member 30 without departing from the spirit and scope of the present invention.

Construction and operation of the retracting and dissecting apparatus according to the present invention structured as described above will be described in detail as follows.

First, a surgeon incises some portion between dermal tissue 100 and spongy tissue 70 at the rear end of the glans penis of the patient, and then, inserts the dissecting portions 12 and 22 into the incised portion with a separation between the plate-shaped dissecting portions 12 and 22 narrowed.

The first and second dissecting members 10 and 20 are provided at one end thereof with grips 11 and 21, respectively.

The surgeon can control the separation between the dissecting portions 12 and 22 of the first and second dissecting members 10 and 20 by adjusting the separation between the grips 11 and 21 with fingers inserted into the grips 11 and 21.

Each of the dissecting portions 12 and 22 of the first and second dissecting members 10 and 20 generally has a plate shape.

The plate-shaped dissecting portions 12 and 22 prevent a dissected portion from being damaged, as compared with the conventional blade-shaped dissecting portions.

More specifically, the plate-shaped dissecting portion 12 has an arc shape, as seen from the outside of the first dissecting member 10, thereby providing a curved surface having a predetermined curvature of radius, whereas the plate-shaped dissecting portion 22 has a flat surface.

As such, with different structures of the dissecting portions 12 and 22, the dermal tissue 100 can be more safely dissected from the spongy tissue 70 of the penis, by the surgeon using the retracting and dissecting apparatus according to the present invention.

FIG. 4 is a diagram illustrating a process of dissecting the dermal tissue 100 from the spongy tissue 70 of the penis using the plate-shaped dissecting portions 12 and 22.

The plate-shaped dissecting portion 12 having the curved surface contacts the spongy tissue 70 of the penis having an approximately circular cross section, while the plate-shaped dissecting portion 22 having the flat surface contacts the dermal tissue 100.

In this state, the dermal tissue 100 can be dissected from the spongy tissue 70 of the penis by adjusting the separation between the dissecting portions 12 and 22.

Here, these features of the dissecting portions 12 and 22 allow easy dissection, and prevent the dissected portion from being damaged during the dissection process.

Meanwhile, during the dissection process, the surgeon handles the apparatus of the invention in a state wherein the latching member 50 is pulled toward the grip 21 with one finger (of the right or left hand), which is not inserted into the grip.

In such a handling state, the latching member 50 is protruded in an arc shape to the first dissecting member 10 by the elastic member 60, and provides a restitution force to compress the latching member 50 toward the holding member 40 having a plurality of saw tooth-shaped protrusions on the outer surface of the arc-shaped holding member 40.

If the surgeon does not pull the latching member 50 toward the grip 21, the retracting and dissecting apparatus is in a ‘fixed’ state wherein the surgeon cannot handle the grips 11 and 21. Thus, in order to handle the grips 11 and 21, the surgeon must pull the latching member 50 toward the grip 21, locating the retracting and dissecting apparatus in a ‘released’ state.

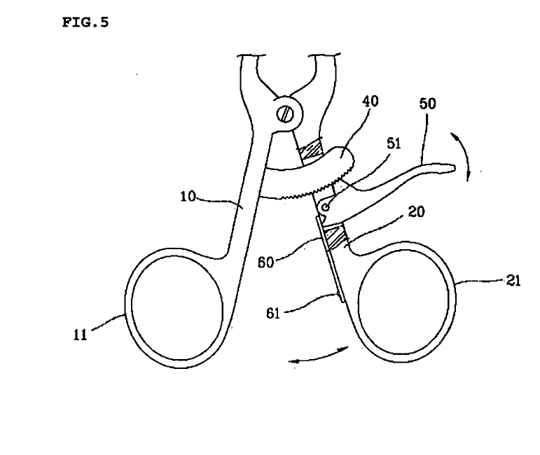

FIG. 5 is a detail view illustrating the holding member 40, the latching member 50, and the elastic member 60 of the retracting and dissecting apparatus of the present invention.

The holding member 40 has one end fixedly coupled to the first dissecting member 10, and the other end inserted into the hole, opening or slot penetrated through the second dissecting member 10, so that the plurality of saw tooth-shaped protrusions of the holding member 40 is provided towards the grip 21 of the second dissecting member 10.

The holding member 40 has an arc shape bent downward in order to allow the first and second dissecting members 10 and 20 to rotate in a state of being coupled to each other by the fastening member 30.

The arc-shaped holding member 40 is provided, on the outer surface thereof, with the plurality of saw-tooth shaped protrusions, which act to hold the latching member 50 at a desired location, thereby maintaining an angle between the first and second dissecting members 10 and 20.

The latching member 50 has a semicircular body, and a protruded elastic force-transmitting portion to which the elastic force is transmitted from the grip 21 and the elastic member 60. The body of the latching member 50 is fixed to the second dissecting member 20 by a rotational shaft 51 of the body.

The body of the latching member 50 is provided, at a predetermined portion, with one or more protrusions, which contact the protrusions of the holding member 40 to prevent the holding member 40 from moving.

The elastic member 60 is attached to an inner surface of the second dissecting member 20 by a screw 61 or other appropriate fastening means.

The elastic member 60 provides an elastic force allowing the latching member 50 to closely contact the holding member 40. Accordingly, when pulling the latching member 50 toward the grip 21, some portion of the elastic member 60 is separated from the inner surface of the second dissecting member 20, thereby providing the restitution force. In this state, when the surgeon releases the latching member 50, the restitution force created in a separated state causes the latching member 50 to rotate and closely contact the holding member 40.

The surgeon dissects the portion between the dermal tissue 100 and the spongy tissue 70 until the suspensory ligament 90 is exposed, using the retracting and dissecting apparatus according to the present invention.

Then, with the dissecting portions 12 and 22 separated from each other, the surgeon releases the latching member 50, causing the latching member 50 to closely contact the protrusions of the holding member 40, thereby maintaining the separation between the dissecting portions 12 and 22.

At this time, with the suspensory ligament 90 positioned between the dissecting portions 12 and 22, the surgeon incises some portion of the suspensory ligament 90 exposed to the outside using a scalpel, and then cauterizes.

As described above, even if the surgeon does not forcibly hold the dissecting portions 12 and 22 apart during incision of the suspensory ligament 90, the separation of the dissecting portions 12 and 22 is fixed, maintaining a dissected state of the dermal tissue 100 and the spongy tissue 70, thereby allowing easy and safe incision of the suspensory ligament 90.

Accordingly, without an additional retracting apparatus or an assistant, the retraction of the latching member is allowed in a state wherein the dermal tissue 100 and the spongy tissue 70 are dissected.

As such, since the additional retracting apparatus is not required in the present invention, the dissected portion can be prevented from being damaged, and the surgical operation can be more easily performed.

Furthermore, since each of the dissecting portions 12 and 22 has the plate shape, only the suspensory ligament 90 is incised with the scalpel, and the dissected portion is prevented from being damaged by the scalpel, thereby increasing safety of the surgical operation.

A rubber pack is attached to an outer surface of each of the dissecting portions 12 and 22 to prevent the dissected portion from being damaged, and such a modification of the present invention is contained in the spirit of the invention.

An example of a concrete operation using the retracting and dissecting apparatus for the surgical operation according to the present invention will now be described as follows.

First, a 2.4˜4.8 cm incision is made into dermal tissue, starting 1 cm below the glans of a penis in the transverse direction, exposing spongy tissue of the penis.

Conventionally, a ring-shaped incision of the dermal tissue, 1 cm below the glans of the penis, is made, thereby exposing the entirety of the spongy tissue. However, penile augmentation according to the present invention can be conducted without entirely incising the dermal tissue.

After exposing the spongy tissue of the penis to the upper layer of Buck's fascia, dissection is performed to the lower portion of the pubic bone using the fingers and the retracting and dissecting apparatus of the present invention.

Subsequently, the suspensory ligament is exposed using the retracting and dissecting apparatus, and some portion of the suspensory ligament is then incised.

The dead space of the incised portion is sutured with aseptic sutures in the transverse direction.

A graft piece of dermal fat taken from the hip is fixed to the Buck's fascia, and the incised portion is sutured, completing the operation.

By the method as described above, minimal incision penile augmentation, in which about 180° incision of the dermal tissue is made without making a full ring-shaped incision of the dermal tissue, can be conducted.

The minimal incision penile augmentation of the present invention utilizes a small incision, thereby reducing suturing time, operation time, and side effects, such as inflammation.

As apparent from the above description, according to the present invention, the dissecting portions have the plate shape, preventing the dissected portion of the patient from being damaged, and particularly, depending on a dissecting position, the dissecting portions have the curved surface and the planar surface, respectively, thereby ensuring a more safe dissection.

Furthermore, the retracting and dissecting apparatus for the surgical operation is provided with the means for maintaining the separation between the dissecting portions, so that other surgical operations can be performed through the dissected portion with the separation between the dissecting portions more easily maintained, thereby allowing an easy surgical operation.

When performing other surgical operations, the plate-shaped dissecting portions prevent the dissected portion adjacent to the operating portion from being damaged, thereby enhancing the credibility of the surgical operation.

It should be understood that the embodiments and the accompanying drawings as described above have been described for illustrative purposes only and the present invention is limited by the following claims. Further, those skilled in the art will appreciate that various modifications, additions and substitutions are allowed without departing from the scope and spirit of the invention as set forth in the accompanying claims.

1. A retracting and dissecting apparatus comprising:
a first dissecting member provided at one end thereof with a grip, at the other end thereof with a first plate-shaped dissecting portion, and at the center thereof with a fastening hole;
a second dissecting member provided at one end thereof with another grip, at the other end thereof with a second plate-shaped dissecting portion, and at the center thereof with another fastening hole;
a fastening member to join the fastening holes of the first and second dissecting members to each other such that the first and second dissecting members can rotate in a predetermined range about the center thereof; and
a position holding member positioned between the center of the first and second dissecting members and the grips of the first and second dissecting members to hold the first and second dissecting members such that a location of the first and second dissecting members does not change unless a user so desires.

2. The apparatus as set forth in claim 1 wherein the position holding member includes an arc-shaped holding member having one end joined to an inner surface between the grip and the center of the first dissecting member while protruding upwardly to the second dissecting member and having the other end penetrating a hole between the grip and the center of the second dissecting member such that the first and second dissecting members can smoothly rotate in a predetermined range, the arc-shaped holding member having a plurality of protrusions on an outer surface of the arc-shaped holding member, a latching member rotatably provided between the hole and the grip of the second dissecting member and engaging with the protrusions of the arc-shaped holding member to hold the first and second dissecting members such that the location of the first and second dissecting members is not changed, and an elastic member coupled to the inner surface of the second dissecting member to exert an elastic force to cause the latching member to closely contact the arc-shaped holding member.

3. The apparatus as set forth in claim 1 wherein one of said first and second plate-shaped dissecting portions includes a curved surface, and one of said first and second plate-shaped dissecting portions includes a planar surface.

4. The apparatus as set forth in claim 1 wherein each of the first and second plate-shaped dissecting portions includes a rubber pack attached to an outer surface thereof.

5. The apparatus as set forth in claim 1 wherein one of the first and second plate-shaped dissecting portions has a curved surface, and one of the first and second plate-shaped dissecting portions has a planar surface, and wherein each of the dissecting portions is provided with a rubber pack attached to an outer surface thereof.

6. Minimal incision penile augmentation comprising the steps of:
incising some portion of dermal tissue a predetermined distance below the glans of a penis such that a semicircular section of spongy tissue of the penis can be exposed with respect to the center of the spongy tissue of the penis;
dissecting the dermal tissue and the spongy tissue of the penis using a retracting and dissecting apparatus according to claim 1 such that the suspensory ligament can be exposed; and
incising some portion of the suspensory ligament exposed between a first plate-shaped dissecting portion and a second plate-shaped dissecting portion of the retracting and dissecting apparatus, the first plate-shaped dissecting portion and the second plate-shaped dissecting portion being separated from each other and held at a separated state.

7. A retracting and dissecting apparatus comprising:
a first dissecting member having a grip associated with one end portion thereof and a first plate-shaped dissecting portion associated with an opposite end portion thereof;
a second dissecting member having a grip associated with one end portion thereof and a second plate-shaped dissecting portion associated with an opposite end portion thereof;
means for pivotally attaching said first and second dissecting members, said first and second dissecting members being rotatable in a predetermined range about said pivot means; and
a position holding member for holding said first and second dissecting members at a particular location relative to each other.

8. The apparatus as set forth in claim 7 wherein said position holding member includes an arc-shaped member having one end portion thereof attached to said first dissecting member and protruding upwardly towards said second dissecting member and having its opposite end portion extending through an opening associated with said second dissecting member such that the first and second dissecting members can smoothly rotate in a predetermined range, said arc-shaped member having a plurality of protrusions on an outer surface thereof, and a latching member rotatably mounted to said second dissecting member, said latching member engaging the protrusions associated with said arc-shaped member to hold the first and second dissecting members at a particular location relative to each other.

9. The apparatus as set forth in claim 8 including an elastic member coupled to said second dissecting member for exerting an elastic force to cause the latching member to closely contact the arc-shaped member.

10. The apparatus as set forth in claim 7 wherein one of said first and second plate-shaped dissecting portions includes a curved surface, and wherein one of said first and second plate-shaped dissecting portions includes a planar surface.

11. The apparatus as set forth in claim 10 wherein each of said first and second plate-shaped dissecting portions includes a rubber pack attached to an outer surface thereof.

12. Minimal incision penile augmentation comprising the steps of:
incising some portion of dermal tissue a predetermined distance below the glans of a penis such that a semicircular section of spongy tissue of the penis can be exposed with respect to the center of said spongy tissue;
dissecting the dermal tissue and the spongy tissue of the penis using a retracting and dissecting apparatus according to claim 7 such that the suspensory ligament can be exposed; and
incising some portion of the suspensory ligament exposed between a first plate-shaped dissecting portion and a second plate-shaped dissecting portion of the retracting and dissecting apparatus, said first and second plate-shaped dissecting portions being separated from each other and held at a separated state.
